SEQUENCE LISTING (2 sequences)

SEQ ID NO: 1 - DNA, 1548 bp, Pimelobacter simplex
atggactgggcagaggagtacgacgtactggtggcgggctccggcgccggcggcatggccgggacctacaccgcggcccgcgaggggctcagcgtgtgcctggtcgaggccggggacaagttcggcgggacgaccgcctactccggcggcggtggggcctggttccccgcgaacccggtgctgctgcgggcgggcaccgacgacacgatcgaggacgctctcgagtactaccgagcggtcgtcggcgaccgcacccccgcggacctgcaggagacctacgtccgcggcggcgccggcctggtcgcctacctcgaggaggacgaccacttctccttcgagtcctacccgtggccggactacttcggcgacgcccccaaggcccgtcgcgacggccagcggcacatcatcccgacgccgctgccggtgccctccgcacccgagctgcgcgaggtggtccgcgggccgctcgacaacgaccggctcggcacgccgcagcccgacgacctgttcatcggcggacgggcgctcgtcgcccgcttcctgaccgcgctcgcgacctacccccacgccacgctcgtgcgcgagaccgcactggccgagctcgtcgtcgaggacggcgtcgtggtcggcgcgatcgtcgagaccgacggcgtccgccgcgcgatccgggcccgccgcggcgtcctcctggccgcgggcggcttcgaggccaatgacgagctccgccagaagtacggcgtccccggcgtcgcgcgcgacacgatgggcccgccgaccaacgtcggcgccgcgcaccaggccgcgatcgcggtcggcgccgacaccgacctgatgggcgaggcctggtggtcccccgggctgacccaccccgacggacgatcggcgttcgcgctctggttcaccggcggcatcttcgtcgacggcgccggccggcgcttcgtcaacgagtcggcgccgtacgaccggctcggccgcgccgtcatcgaccacctcaccgagggcggcgtcaccccgcggtactggatggtctacgaccacaaggagggctcgatccccccggtgcgcgccaccaacgtctcgatggtcgacgaggagcagtacgtcgccgcgggcctgtggcacaccgccgacacgctgcccgagctggccgcgctgatcggcgtccccgccgacgcgctggtcgccacggtcgcgcgcttcaacgagctcgtcgccgacgggtacgacgcggacttcggccgcggcggcgaggcctacgaccggttcttctccggcggcgagccgccgctggtgagcatcgacgaggggccgttccacgcggccgccttcggcatctccgacctcggcaccaagggcgggctgcgcaccgacacgtccgcgcgcgtgctgaccgcggacggcacgccgatcgggggcctctacgcagccggcaatacgatggcggcgccgagcggcaccacctacccgggcggtggcaacccgatcgggacaagcatgctcttcagccacctcgccgtgcggcacatgggcaccgaggacgcgcgatga

SEQ ID NO: 2 - protein, 515 aa, Pimelobacter simplex
MDWAEEYDVLVAGSGAGGMAGTYTAAREGLSVCLVEAGDKFGGTTAYSGGGGAWFPANPVLLRAGTDDTIEDALEYYRAVVGDRTPADLQETYVRGGAGLVAYLEEDDHFSFESYPWPDYFGDAPKARRDGQRHIIPTPLPVPSAPELREVVRGPLDNDRLGTPQPDDLFIGGRALVARFLTALATYPHATLVRETALAELVVEDGVVVGAIVETDGVRRAIRARRGVLLAAGGFEANDELRQKYGVPGVARDTMGPPTNVGAAHQAAIAVGADTDLMGEAWWSPGLTHPDGRSAFALWFTGGIFVDGAGRRFVNESAPYDRLGRAVIDHLTEGGVTPRYWMVYDHKEGSIPPVRATNVSMVDEEQYVAAGLWHTADTLPELAALIGVPADALVATVARFNELVADGYDADFGRGGEAYDRFFSGGEPPLVSIDEGPFHAAAFGISDLGTKGGLRTDTSARVLTADGTPIGGLYAAGNTMAAPSGTTYPGGGNPIGTSMLFSHLAVRHMGTEDAR

(19) United States
(12) Patent Application Publication
(10) Pub. No.: US 2007/0015236 A1
(43) Pub. Date: Jan. 18, 2007

(54) Process of preparing prednisolone devoid of steroid-derived impurities using recombinant E. coli transformed with A delta¹-dehydrogenase gene

(76) Inventors: Albert Fliss, Mahopac, NY (US); Horacio Bach, Vancouver (CA)

Correspondence Address: TARO PHARMACEUTICALS U.S.A., INC.;C/O VENABLE LLP, P.O. BOX 34385, WASHINGTON, DC, 20043-9998, US

(21) Appl. No.: 11/398,322

(22) Filed: Apr. 5, 2006

Related U.S. Application Data
(60) Provisional application No. 60/668,410, filed on Apr. 5, 2005.

(51) Int. Cl. C12P 33/00; C12N 9/04; C12N 1/21

(52) U.S. Cl. 435/52; 435/190; 435/252.33; 435/252.34

(57) ABSTRACT

The present invention relates to a process of using recombinant E. coli having a Δ¹-dehydrogenase gene to prepare prednisolone. Specifically, the present invention relates to a fermentation process of using recombinant E. coli containing a Δ¹-dehydrogenase gene to prepare prednisolone that is devoid of steroid-derived impurities.

### CROSS REFERENCE TO RELATED APPLICATION

This application claims the benefit under 35 U.S.C. §119(e) of the U.S. Provisional Application No. 60/668,410 filed Apr. 5, 2005, the disclosure of which is incorporated by reference in its entirety herein.

### FIELD OF THE INVENTION

The present invention relates generally to a process of using a genetically modified microorganism for the manufacture of a useful steroid. More specifically, the present invention relates constructing an E. coli strain by genetic insertion of a Δ¹-dehydrogenase gene capable of converting hydrocortisone into prednisolone that is devoid of steroid-derived impurities.

### BACKGROUND OF THE INVENTION

Prednisolone (11β, 17α, 21-trihydroxypregna-1,4-diene-3,20-dione) is a synthetic steroid that is of significant importance in the treatment of inflammatory diseases such as rheumatoid arthritis and asthma. Chemical synthesis to prepare prednisolone involves multiple reaction steps and is not economically feasible. Use of microorganisms for steroid bioconversion is known. For example, U.S. Pat. No. 2,837,464 describes the 1-dehydrogenation of steroids by addition of steroid substrate (e.g., hydrocortisone) to a fermentation culture of Corynebacterium simplex. U.S. Pat. No.3,056,146 discloses Bacterium cyclooxydans as a 1-dehydrogenator for bioconversion. This microbial bioconversion of steroids is a reaction of commercial importance. The Δ¹-dehydrogenation is mediated by the Δ¹-dehydrogenase (EC 1.3.99.4) present in the cells (e.g., Bacterium cyclooxydans or Nocardioides simplex (formerly known as Arthrobacter simplex or Corynebacterium simplex; ATCC 6946)). The Δ¹-dehydrogenase introduces double bonds into ring A at the positions C₁-C₂ of the hydrocortisone and forms prednisolone. Herzog et al. discloses the insertion of a double bond at the 1,2 position of hydrocortisone causes the resulting steroid of prednisolone to have enhanced potency (Science, vol. 121. p. 176, 1955).

With respect to the bioconversion of hydrocortisone to prednisolone, U.S. Pat. No. 4,839,282 discloses a fermentation process of using Nocardioides simplex in the presence of cobalt ions in an effort to minimize steroid metabolism and feedback inhibitions. U.S. Pat. Nos. 4,524,134 and 4,704,358 further disclose an improved fermentation process to prepare prednisolone using air-dried or heat-dried microbial cells. While these fermentation processes use native bacterial cells to produce prednisolone and enhance its conversion rate, they concomitantly produce a variety of steroid-derived impurities, often in the amounts exceeding the U.S. Pharmacopeia (USP) requirement (e.g., about 5-20%). The presence of these steroid-derived impurities in prednisolone exceeding the USP requirement is undesirable. For example, Arinbasarova et al. reported the production of 20β-hydroxy derivatives during hydrocortisone transformation to prednisolone. (J. Steroid Biochem. 23(3): 307-312, 1985) Gorog et al. discloses three other steroid-derived impurities during fermentation production of prednisolone. (J. of Pharmaceutical & Biomedical Analysis 181: 511-523, 1998) These other steroid-derived impurities include 11α-hydroxy, 11-oxo and 1 1-deoxy of 1,4-diene-3-oxo-11β-hydroxy steroids.

The oxidation/reduction reactions involved in the steroid-derived impurities production is thought to be mediated by the native enzymes present in the wild-type Nocardioides simplex bacteria. These native enzymes may include 3-ketosteroid-1-en-dehydrogenase, 20β-hydroxysteroid dehydrogenase and 3-ketosteroid, 1-2-reductase, all of which may act on the substrate (hydrocortisone) as well as on the product (prednisolone) to produce 20β-hydrocortisone and 20β-prednisolone impurities, respectively. Exact responsible enzymes are presently unknown. The involvement of P450 complex enzyme system, if any, is unclear. Nevertheless, these steroid-derived impurities, once formed, are known to be difficult to separate from the final product (i.e., prednisolone) because of their structural similarity (See, e.g., Arinbasarova et al., J. Steroid Biochem. 23(3): 307-312, 1985; Gorog et al., J. of Pharmaceutical & Biomedical Analysis 181: 511-523, 1998).

USP requires a contamination level of no greater than 0.6% of any individual steroid-derived impurity and no greater than 2.0% of total steroid-derived impurities in prednisolone. Attempts have been made to reduce the steroid-derived impurity levels of prednisolone during the Nocardioides simplex fermentation U.S. Pat. No. 4,749,649 discloses the addition of an exogenous electron carrier to achieve a faster rate of bioconversion. An enhanced bioconversion rate often is accompanied by an increased production of steroid-derived impurities. The '649 patent discloses the use of an oxygen radical scavenger (e.g., catalase or superoxide dismutase) to decrease the formation of undesirable side-products (e.g., steroid-derived impurities) during the bioconversion of hydrocortisone to prednisolone. Kloosterman et al. discloses adding an electron acceptor (i.e., phenazine methosulfate) in immobilized Arthrobacter simplex cells (Kloosterman et al., Biotechnologv Letters 7(l):25-30). These maneuvers are costly as Kloosterman et al. expressly states that the use of artificial electron acceptors is expensive.

While most prior art fermentation processes utilize native cells such as Nocardia, Mycobacterium and Streptomyces, there is no presently available information regarding use of a recombinant E. coli based expression system to prepare prednisolone.

There remains a great need for a fermentation process to prepare prednisolone that is devoid of steroid-derived impurities. It would be desirable to develop a recombinant approach to prepare prednisolone that is devoid of steroid-derived impurities.

### SUMMARY OF THE INVENTION

The present invention provides a process of constructing a recombinant host, which is capable of producing prednisolone when fermentation is performed in the presence of hydrocortisone.

One object of the present invention is to provide a process for preparing prednisolone, such method utilizing a novel recombinant E. coli as the producer of prednisolone.

The present invention provides a process for preparing prednisolone, comprising the steps of:
(a) preparing an E. coli transformed with a recombinant DNA molecule which is a plasmid containing a nucleic acid sequence for Δ¹-dehydrogenase gene;
(b) culturing the transformed E. coli in a fermentation medium containing hydrocortisone; and
(c) recovering prednisolone from the fermentation medium, wherein the recovered prednisolone is devoid of steroid-derived impurity.

Another object of the present invention is to insert a Δ¹-dehydrogenase gene into a host bacterial microorganism (i.e., E. coli) that functions to introduce double bonds into ring A at the positions C₁-C₂ of the hydrocortisone.

Preferably, the present invention relates to transformation of E. coli cells with a recombinant DNA molecule containing a plasmid that comprises the Δ¹-dehydrogenase gene from the microorganism Nocardioides simplex ATCC 6946. Preferably, the nucleic acid sequence of the Δ¹-dehydrogenase gene is set forth in SEQ ID NO: 1.

The present invention provides a process for preparing an E. coli transformed with a recombinant DNA that encodes a Δ¹-dehydrogenase polypeptide (SEQ ID NO: 2; Accession Number: D37969; Protein ID=BAA07186.1). Preferably, the amino acid sequence of the Δ¹-dehydrogenase is set forth in SEQ ID No: 2.

Preferably, the recombinant DNA molecule is isolated from Nocardioides simplex (Accession number: D37969).

Preferably, the E. coli is a microorganism selected from the group consisting of BL-21 (DE3), BL-21, DH5α, XL1-Blue XL1-series, JM series and the like. More preferably, the bacterial cell is BL-21 (DE3).

Preferably, the fermentation medium contains hydrocortisone at a concentration of about 0.1 to about 30 grams/L. Preferably, the fermentation medium contains hydrocortisone at a concentration of about 1 to about 4 grams/L. More preferably, the fermentation medium contains hydrocortisone at a concentration of about 2 grams/L.

Another object of the present invention is to provide a process for preparing prednisolone that is devoid of any steroid-derived impurities. Preferably, the recovered prednisolone contains less than about 1% total steroid-derived impurity. Preferably, the recovered prednisolone contains less than about 0.5% total steroid-derived impurity. Preferably, the recovered prednisolone contains less than about 0.1% total steroid-derived impurity. More preferably, the recovered prednisolone contains less than about 0.01% total steroid-derived impurity.

The steroid-derived impurity is at least one compound selected from the group consisting of 20β-hydroxy derivative of prednisolone, 11β-hydroxy derivative of prednisolone, 11-oxo derivative prednisolone and 11-deoxy of prednisolone. Preferably, the steroid-derived impurity is 20β-hydroxy derivative of prednisolone. Preferably, the steroid-derived impurity is 11α-hydroxy derivative of prednisolone. Preferably, the steroid-derived impurity is 11-oxo derivative prednisolone. Preferably, the steroid-derived impurity is 11-deoxy of prednisolone.

The present invention further provides prednisolone having less than about 1% total steroid-derived impurity prepared by a process which comprises the steps of:
(a) preparing an E. coli transformed with a recombinant DNA molecule which is a plasmid containing a nucleic acid sequence set forth in SEQ ID No: 1;
(b) culturing the transformed E. coli in a fermentation medium containing hydrocortisone; and
(c) recovering prednisolone from the fermentation medium.

### BRIEF DESCRIPTION OF THE INVENTION

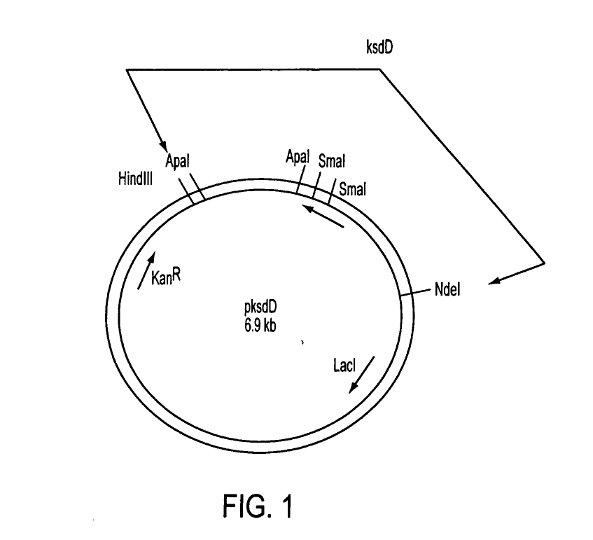

FIG. 1 shows the pet-28b-derived plasmid pksdD which contains the Δ¹-dehydrogenase gene (i.e., ksdD gene) (SEQ ID NO: 1; Accession Number: D37969).

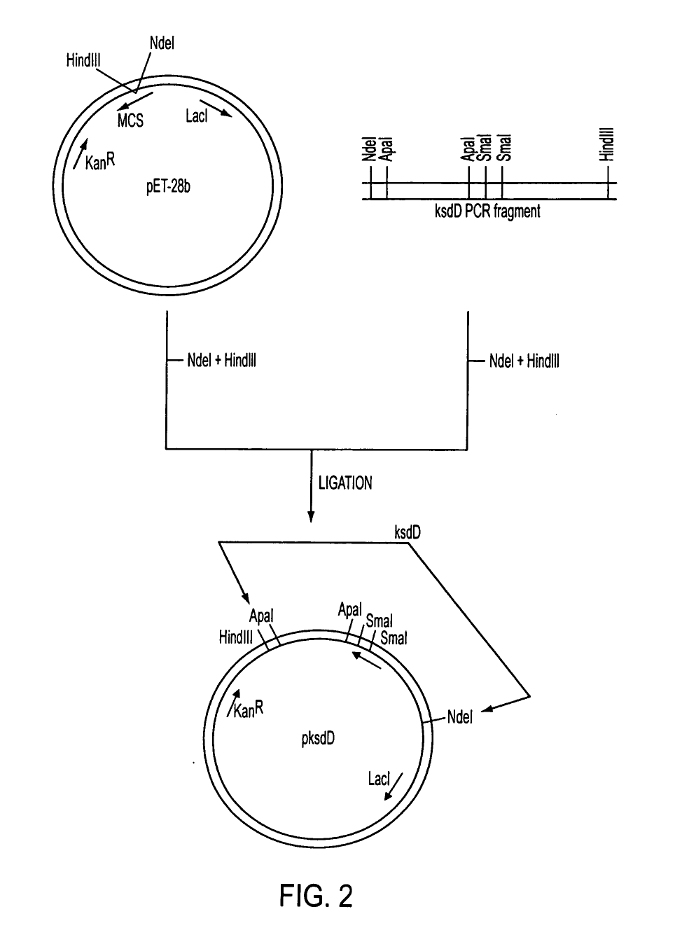

FIG. 2 shows the steps of constructing a plasmid pksdD, which is the pET-28b expression vector containing the ksdD gene.

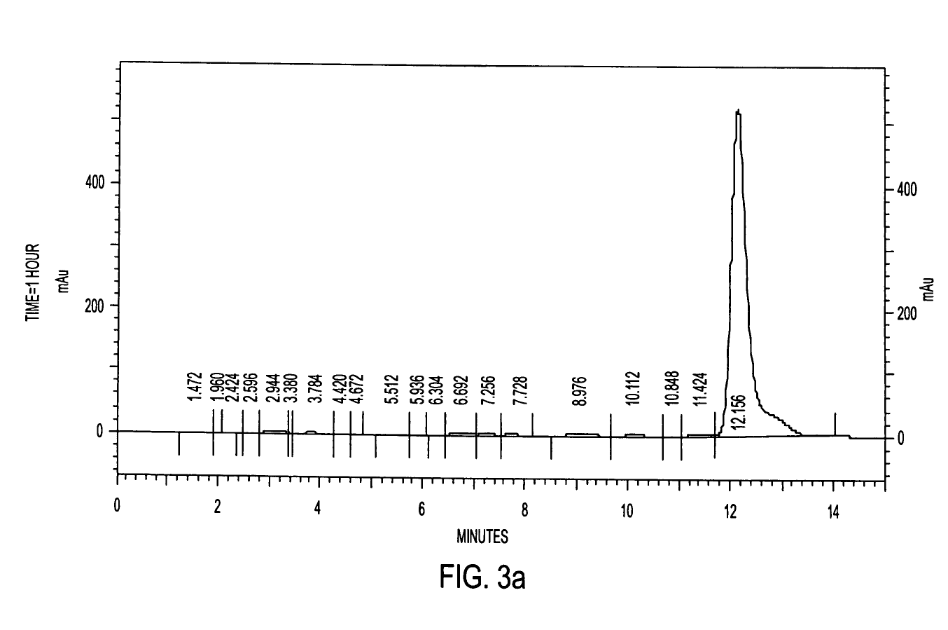

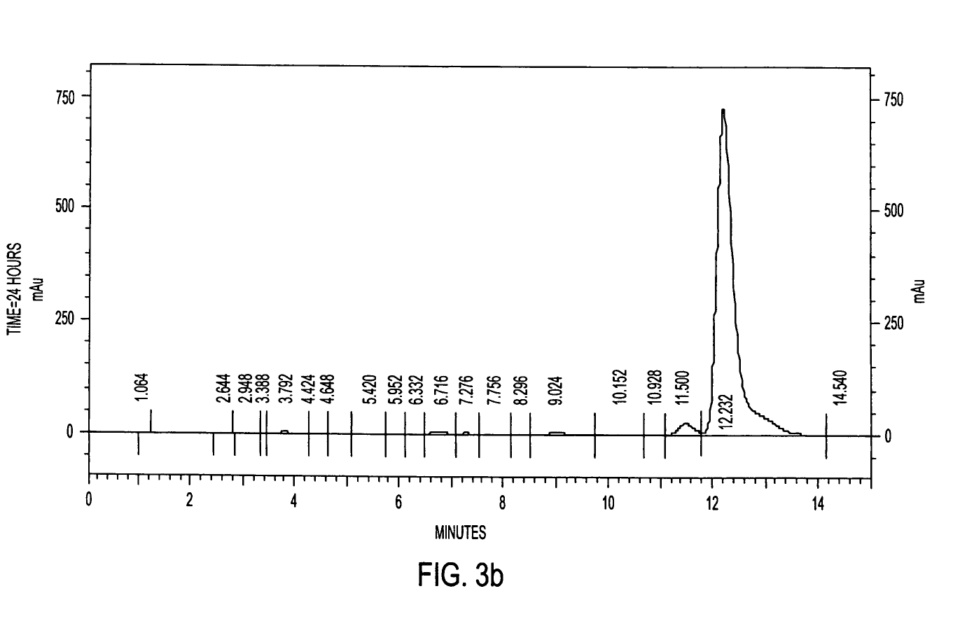

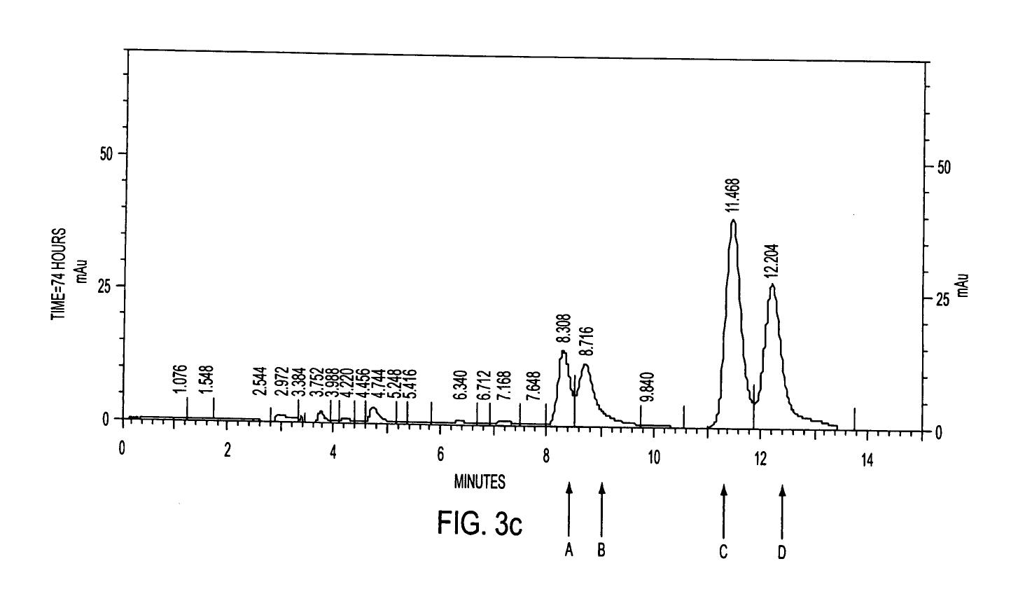

FIG. 3 shows a representative HPLC tracing of prednisolone and steroid-derived impurities from fermentation broth using wild-type Nocardioides simplex (ATCC 6946).

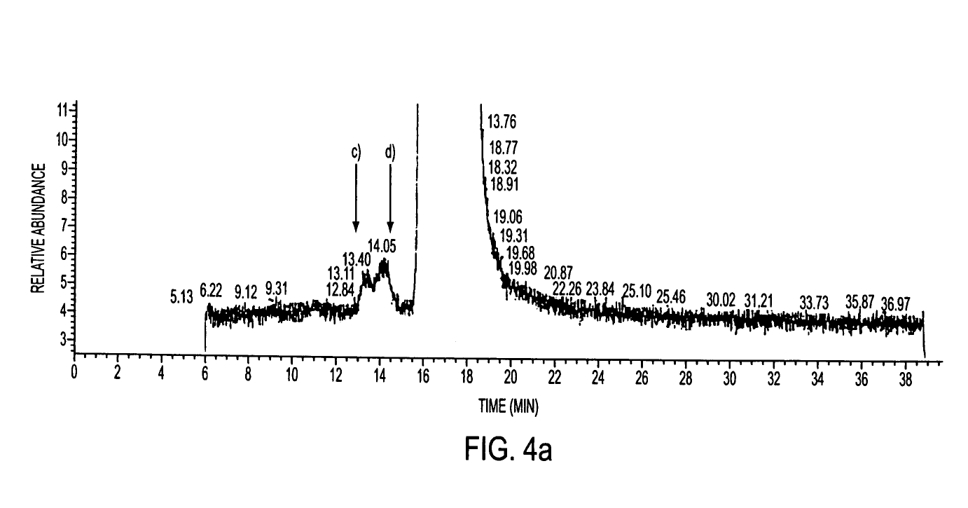

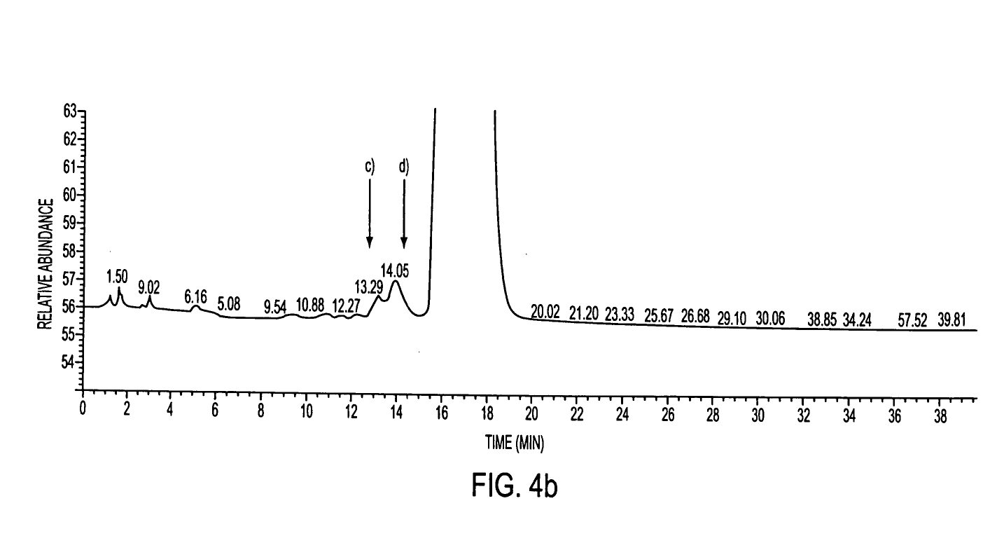

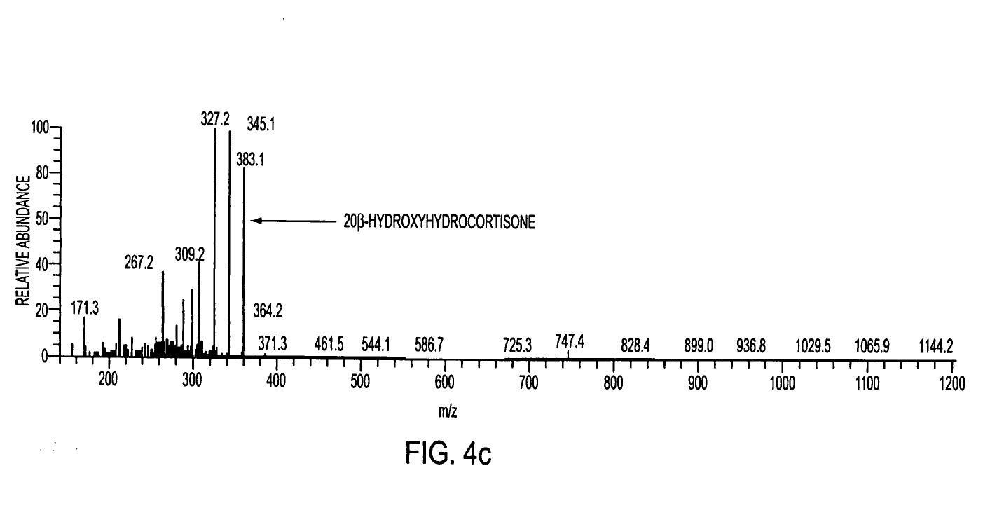

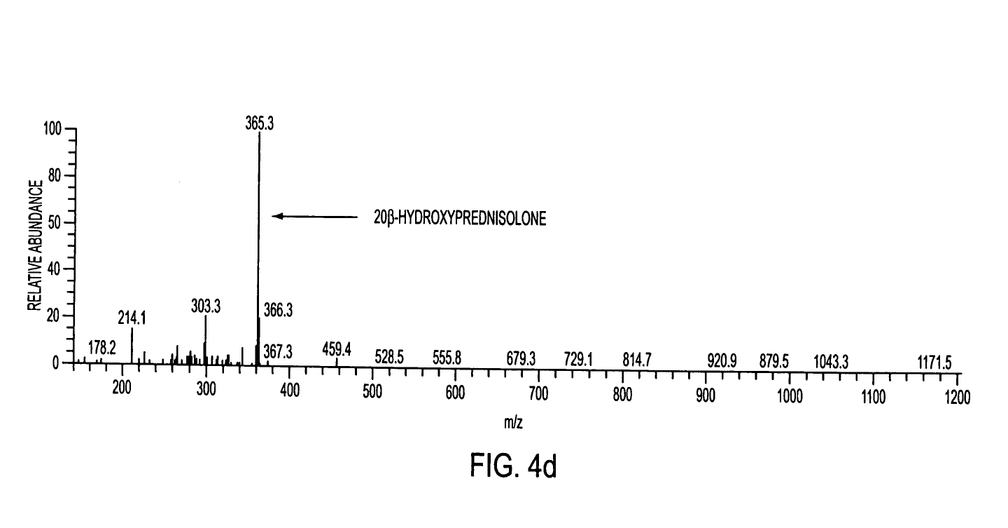

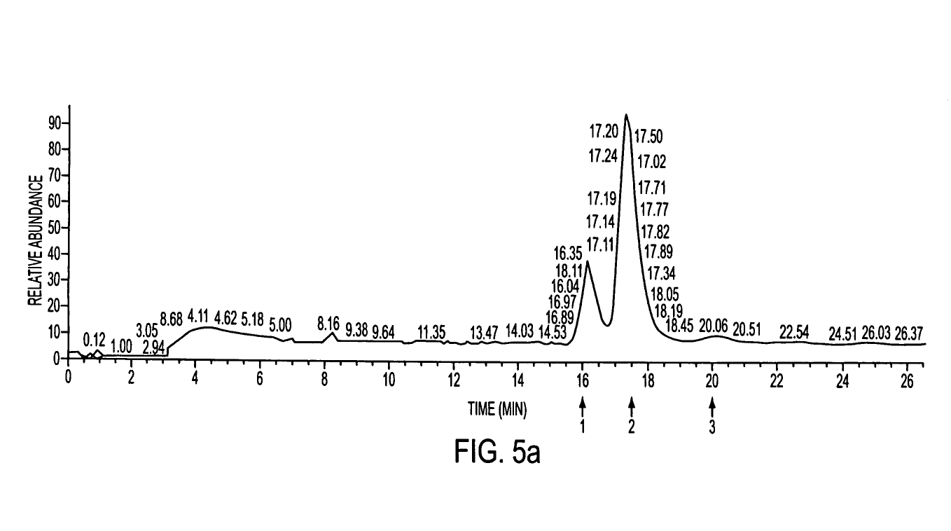

FIG. 4 shows a representative HPLC separation and mass spectrometry analysis (MS) of two steroid-derived impurities (identification of 20β-hydroxyhydrocortisone (4c) and 20β-hydroxyprednisolone (4d)) from fermentation broth using wild-type Nocardioides simplex (ATCC 6946).

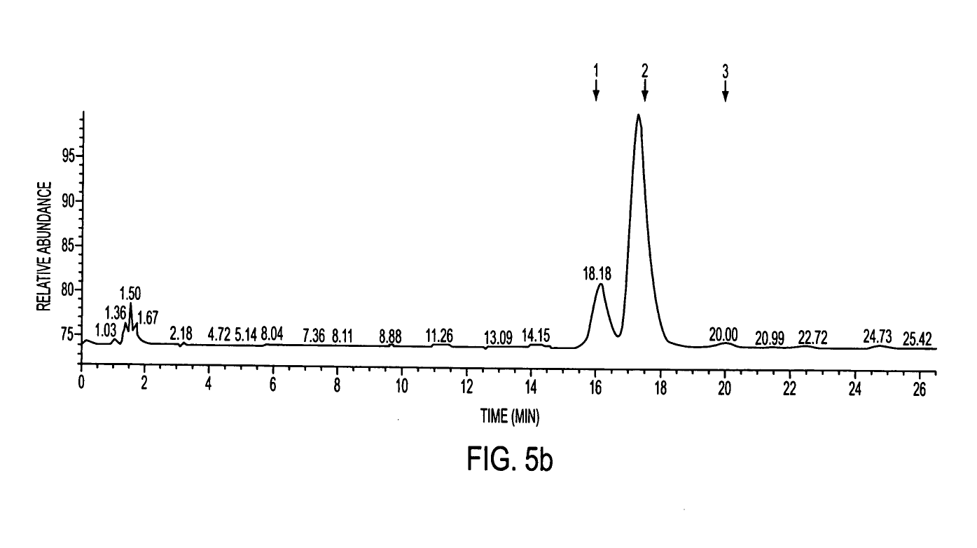

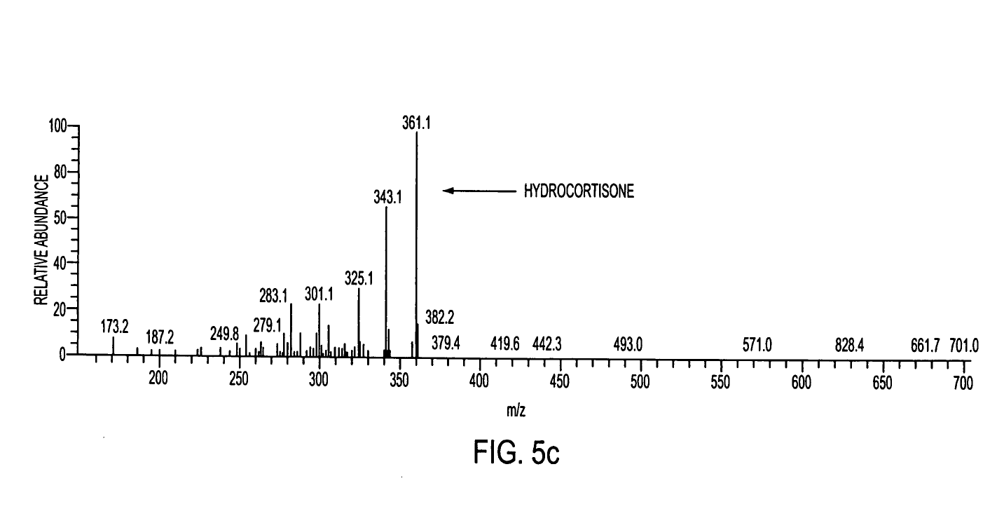

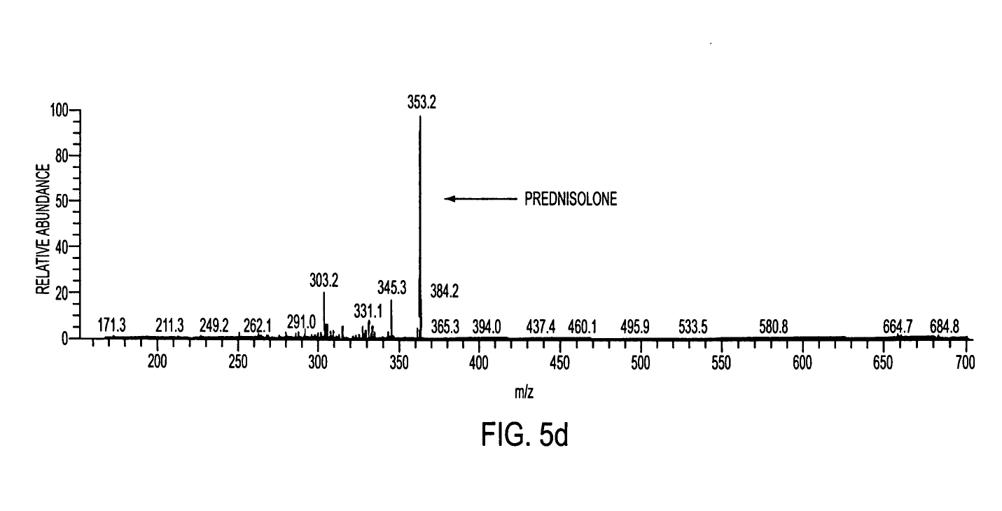

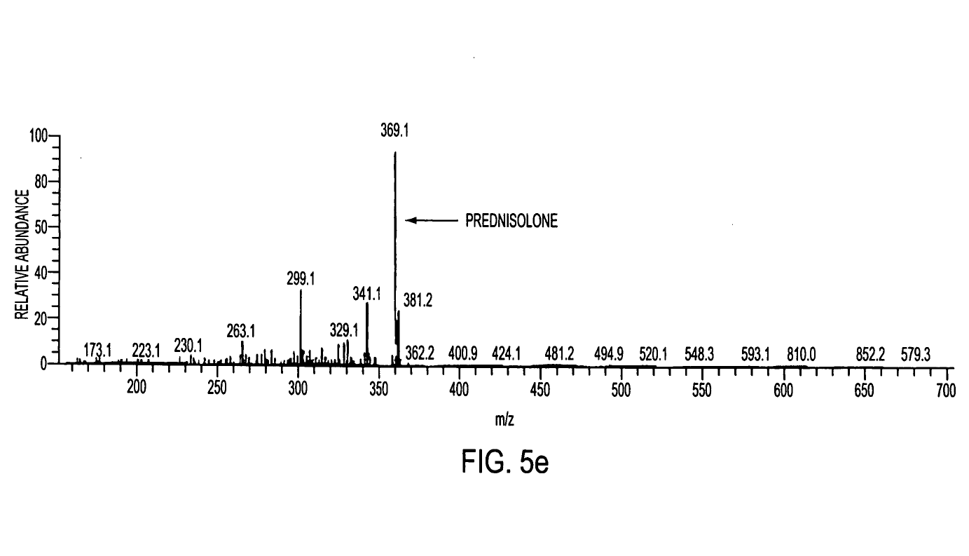

FIG. 5 shows a representative HPLC separation and mass spectrometry analysis (MS) of the substrate (hydrocortisone) (5c), the product (prednisolone) (Sd) and a steroid-derived impurity (identification of 11-oxo-derivative of prednisolone (5e)) from fermentation broth using wild-type Nocardioides simplex (ATCC 6946).

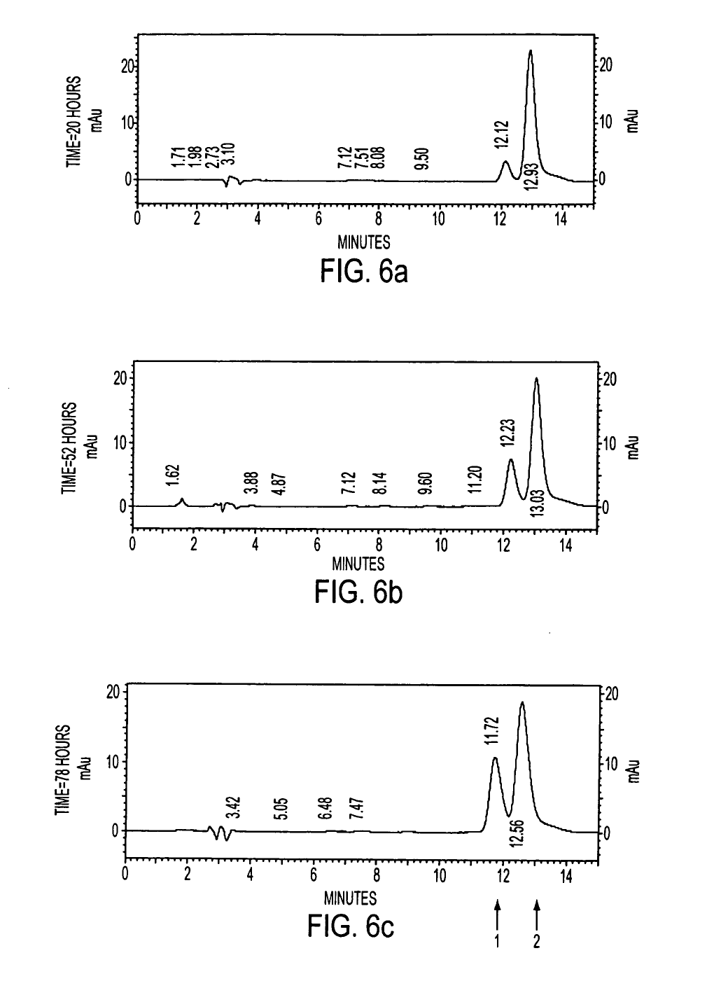

FIG. 6 shows a representative HPLC tracing of the substrate (hydrocortisone) and the product (prednisolone) but absence of steroid-derived impurities from fermentation broth in shake flasks using recombinant E. coli BL-21 (DE3) hosting the ksdD gene (SEQ ID NO. 1; Accession Number: D37969).

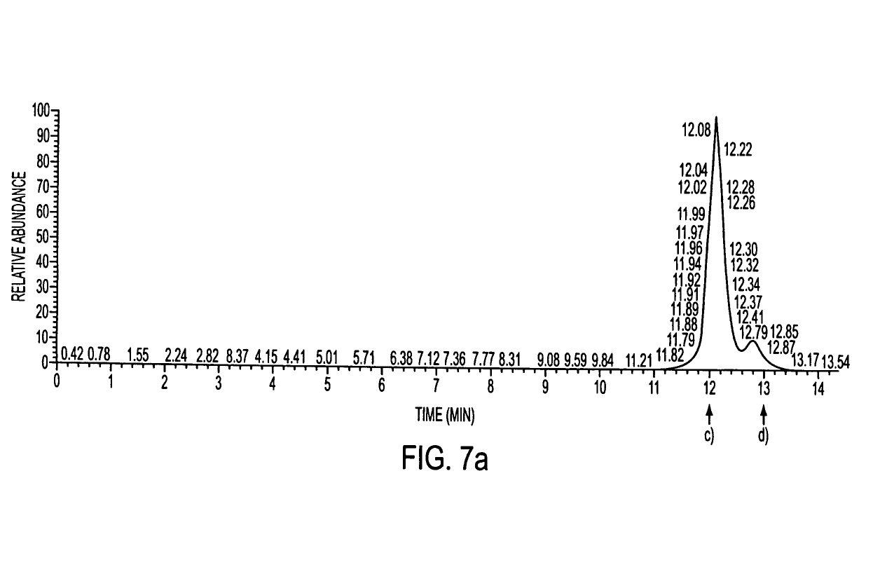

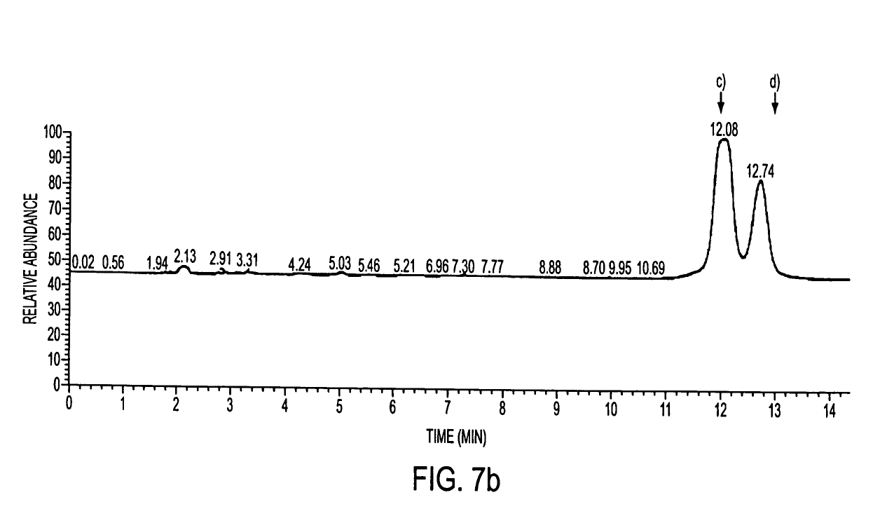

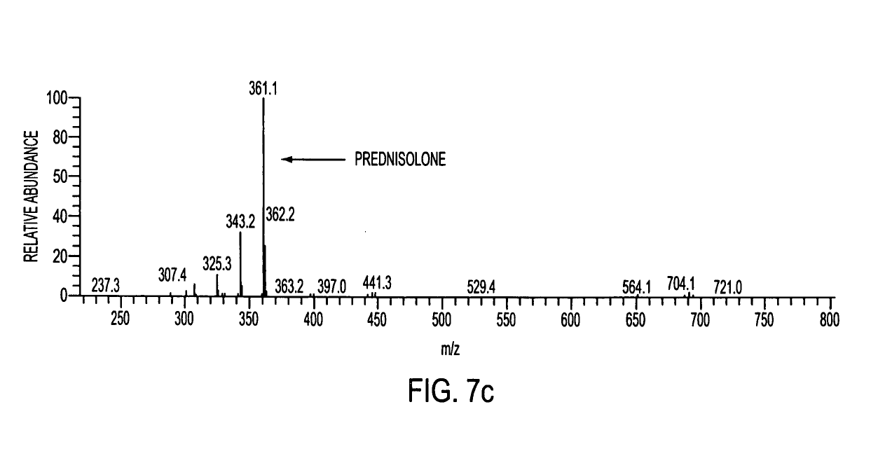

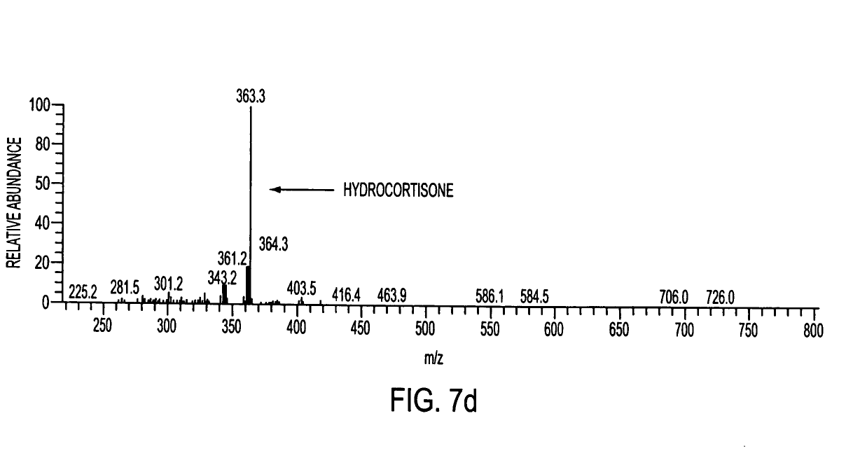

FIG. 7 shows a representative HPLC separation and mass spectrometry analysis (MS) of the substrate (hydrocortisone) (7d) and the product (prednisolone) (7c) but absence of steroid-derived impurities from fermentation broth in a 2L fermentor using recombinant E coli BL-21 (DE3) hosting a ksdD gene (SEQ ID NO: 1; Accession Number: D37969).

### DETAILED DESCRIPTION OF THE INVENTION

Definitions:

As used herein, the term “pksdD” refers to a plasmid containing a ksdD gene; “ksdD” refers to a Δ¹-dehydrogenase gene; “Δ¹-dehydrogenase gene” refers to ksdD gene having a nucleotide sequence as exemplified by SEQ ID NO: 1 (Accession Number: D37969), a conservative change of nucleotide without altering its enzymatic function of adding a double bond into ring A at the positions C₁-C₂ of the hydrocortisone is encompassed; “plasmid vector pET-28b” refers to an E. coli based expression vector; “vector” refers to a piece of DNA that enables one to transfer a foreign autonomously replicating fragment of DNA into a foreign host; “transformation” refers to a process of inserting a plasmid (often containing a foreign gene) into a cell; “HPLC” refers to high performance liquid chromatography; “steroid-derived impurities” refers to undesirable impurities due to modifications of hydrocortisone and prednisolone. The modifications encompass hydroxylation, oxidation, or reduction at various positions on the hydrocortisone and prednisolone. Exemplary steroid-derived impurities include, but not limited to, 20β-hydrocortisone, 20β-prednisolone impurities, 20β-hydroxy derivative of prednisolone, 11α-hydroxy derivative of prednisolone, 11-oxo derivative prednisolone, 11-deoxy of prednisolone and the like. It is to be understood that these modifications may involve several enzymes such as mono-oxygenases, cytochrome P-450 enzymes, reductases and dehydrogenases.

As such herein, the term “devoid of” refers to less than about 2% of total steroid-derived impurities. As required by U.S. Pharmacopoeia, commercial prednisolone contains less than about 2% total steroid-derived impurities.

For the purposes of the present application, “shake flask experiment” refers to a fermentation performed in a Erlenmeyer flask of 250 mL; “fermentor experiment” refers to a fermentation performed in a 2L fermentor.

We have surprisingly found that an E. coli recombinant expression system can convert hydrocortisone into prednisolone. Contrary to yeast cells, E. coli is known to lack the basic machinery required for steroid synthesis. For example, while yeast cells contain enzymes such as adrenodoxin and adrenodoxin reductase (ADX/ADR), E. coli does not contain such enzymes. The ADX/ADR represents an electron recycling system widely thought to be required for many steps in the steroid synthesis pathway. To the best knowledge of the applicants, there has been no report using a recombinant E. coli cell to convert steroids. Accordingly, the present finding that recombinant E. coli can convert hydrocortisone to prednisolone is surprising in light of the fact that E. coli lacks the proper electron recycling system (e.g., adrenodoxin and adrenodoxin reductase (ADX/ADR)).

The use of other microbial cells such as Nocardioides, Pseudomonas and Mycobacterium are all accompanied by steroid-derived impurities. We have surprisingly found that the prednisolone produced by the present recombinant microorganism (i.e., recombinant E. coli expression system) is devoid of contaminants (i.e., steroid-derived impurities). Accordingly, the present recombinant E. coli system constitutes an improved system leading to the production of prednisolone that is devoid of steroid-derived impurities.

It is found that wild-type E. coli does not make prednisolone from hydrocortisone. This observation is in line with the fact that wild-type E. coli lacks the Δ¹-dehydrogenase (EC 1.3.99.4) or other homologous dehydrogenases necessary for the introduction of a double bond into ring A at the positions C₁-C₂ of the hydrocortisone (Genome accession number: NCO00913). Insertion of the ksdD gene into E. coli by recombinant manipulations allows the recombinant microorganism to synthesize prednisolone from hydrocortisone.

It is found that gene insertion of Δ¹-dehydrogenase (EC 1.3.99.4) or other homologous dehydrogenases in E. coli provides satisfactory results in the bioconversion of hydrocortisone to prednisolone without significant production steroid-derived impurities. Without wishing to be bound by a theory, applicants believe that the lack of a P450 system in E. coli may contribute to the improvement. This observation is surprising, in part because there is no established linkage of the P450 complex system in the generation of steroid-derived impurities in microbial cells. The presence of steroid-derived impurities is known to exist in some recombinant microorganisms (such as yeast strains) during the steroid bioconversion (See, e.g., Dragan C. et al. FEMS Yeast Research 5, 621-625, 2005, and Szczebara, F.M., C. et al, Nature Biotechnology, vol. 21, February 2003). Yeast is known to contain the multiple P450 enzyme complex systems.

For purposes of the present invention, it is believed that other microbial cells lacking a P450 system may function equivalently as E. coli. Accordingly, the present invention encompasses the gene insertion of a ksdD gene in a P450-lacking microbial cell and the use of such recombinant microbial cells in preparing prednisolone is equivalent to that of using recombinant E. coli.

According to the present invention, such improved process of producing prednisolone, comprises the steps of:
(a) preparing an E. coli transformed with a recombinant DNA molecule which is a plasmid containing a nucleic acid sequence set forth in SEQ ID No: 1;
(b) culturing the transformed E. coli in a fermentation medium containing hydrocortisone; and
(c) recovering prednisolone from the fermentation medium, wherein the recovered prednisolone is devoid of steroid-derived impurity.

i) Δ¹-Dehydrogenase Gene

The gene coding for Δ¹-dehydrogenase enzyme from various microorganisms can be cloned for use in the present invention. Any microorganism, as far as it exhibits Δ¹-dehydrogenase enzyme activity, can potentially serve as the nucleic acid source for the molecular cloning of the gene coding for Δ¹-dehydrogenase enzyme. A microorganism belonging to the genus Nocardioides and having Δ¹-dehydrogenase enzyme activity is preferred. A preferred example is Nocardioides simplex ATCC 6946.

While not being limited to any particular mode of action, it is thought that the inserted Δ¹-dehydrogenase functions to introduce double bonds into ring A at the positions C₁-C₂ of the hydrocortisone to form prednisolone. The oxidation is accomplished in one-step reaction and by the single enzyme activity of Δ¹-dehydrogenase.

The Δ¹-dehydrogenase enzyme is defined as EC 1.3.99.4 according to the International Union of Biochemistry and Molecular Biology (IUBMB Enzyme Nomenclature). It's synonymous names include 3-oxosteroid-1-dehydrogenase, 1-ene-dehydrogenase, 3-ketosteroid-1-en-dehydrogenase, 3-ketosteroid-Δ¹-dehydrogenase, 3-oxosteroid-Δ¹-dehydrogenase, 4-en-3-oxosteroid: (acceptor)-1-en-oxidoreductase, 3-oxosteroid: (2,6-dichlorphenolindophenol) Δ¹-oxidoreductase, 3-oxo-1-dehydrogenase and Δ¹-steroid reductase.

The DNA containing the gene coding for Δ¹-dehydrogenase enzyme may be obtained by standard procedures known in the art, from a DNA library prepared by cloning chromosomal DNA or fragments thereof, as purified from the microbial cells of the genus Nocardioides, into a suitable vector for propagation of the gene (See, for example, Maniatis et al., 1982, Molecular cloning, A Laboratory Manual, Cold Spring Harbor Laboratory, Cold Spring Harbor, N.Y.; Glover, D. M. (ed.)).

Preferably, the source of the Δ¹-dehydrogenase gene is of bacterial origin. Examples of the source for the Δ¹-dehydrogenase gene include, but are not limited to Nocardioides simplex ATCC 6946, Pseudomonas testeroni, Pseudomonas fluorescens, Mycobacterium smegmatis, and the like. More preferably, the bacterial source is Nocardioides simplex ATCC 6946.

The nucleotide sequence of the preferred bacterial Δ¹-dehydrogenase gene is shown in SEQ. ID. (SEQ ID NO: 1; Accession No. D37969). The amino acid sequence of the encoded Δ¹-dehydrogenase enzyme is shown in SEQ ID NO: 2 (Accession No. BAA07186.1).

Δ¹-dehydrogenase homologues are to be considered functionally equivalent by those skilled in the art. It is considered that Δ¹-dehydrogenase gene homologues that exhibit ≧60% identity at the nucleic acid are functionally indistinguishable. The present invention encompasses Δ¹-dehydrogenase homologues having ≧60% identity and functions to introduce double bonds into ring A at the positions C₁-C₂ of the hydrocortisone to form prednisolone.

ii) Plasmid Vectors

Once the DNA fragments are generated, DNA constructs are prepared using an appropriate cloning and/or expression vector. The Δ¹-dehydrogenase gene is incorporated into a suitable vector that is used to transform a suitable cellular host. Microorganisms known as 1-dehydrogenators and listed in Charney, W. and Herzog, H. 1967. Microbial Transformation of Steroids. Academic press, Inc., NY, may represent a microbial source of 3-oxo-1-dehydrogenase genes.

Preferred plasmid vectors include, but not limited to, the plasmid vector pET-28b (Novagen, Calif.). Other suitable vectors or cloning systems or protein expression systems by which the Δ¹-dehydrogenase is expressed and its dehydrogenase activity manifested may also be used. Plasmid vectors containing inducible promoters are suitable, e.g., tac, araBAD, T7, trp and the like. Examples of commercial available plasmid vectors, cloning and/or expression systems include Impact series, pCAL series, pET expression system series, pGEX series, PinPoint series, pMAL series, Radiance series, Variflex series and the like. Preferably, the plasmid vector is of the pET series. More preferably, the plasmid vector is pET28b. Alternatively, the Δ¹-dehydrogenase gene may be inserted directly in the genome by standard methods.

Incorporation of the gene into a shuttle vector is accomplished by first digesting the plasmid with suitable restriction endonucleases, followed by annealing the gene insert to the plasmid “sticky ends,” and then ligating the construct with suitable ligation enzymes to re-circularize the plasmid. These steps are well known to those skilled in the art and need not be described in detail here. (See, e.g., Sambrook, and Russell (2001), Molecular Cloning, A Laboratory Manual, 3th Ed., incorporated herein by reference for its teaching of vector construction and transformation).

Prior to expression of a heterologous protein in E. coli, the Δ¹-dehydrogenase gene is first sub-cloned into an expression vector that is capable of synthesizing the gene product in E. coli. Accordingly, the expression vector requires an E. coli origin of replication, a selectable marker or antibiotic resistance genes and a promoter to drive expression in E coli. Preferred expression vectors capable of performing this activity are derived from vector backbones of pBR322, puc and m13 series. These expression vector backbones all contain an origin of replication capable of driving DNA replication in E coli. Other expression vectors derived from these backbones would be considered equivalent by one skilled in the art. The present invention encompasses expression vectors derived from pBR322, puc, m13 series that share functional equivalency. Expression vectors include, but are not limited to, pet28 a-c series, pet series, puc series, pBR322 series, m13 series, pGEX series, pMBP series and the like.

T7 promoter is preferred to drive the expression of the Δ¹-dehydrogenase gene in E coli. Other suitable promoters include, but are not limited to, lacZ, tet, tac, araBAD, T7, trp and the like. These suitable promoters would be considered functionally similar by a skilled in the art.

Preferably, a selectable marker or antibiotic resistance gene is needed to maintain the recombinant plasmid in its host stably. Suitable selectable marker includes, but not limited to, trp, met, his and the like. Suitable antibiotic resistance markers include, but not limited to, ampicillin, chloroamphenicol, tetracycline, Neomycin, G418, genticin and the like.

In our specific embodiment, the Δ¹-dehydrogenase gene of Nocardioides simplex (SEQ ID NO: 1) is inserted into the pET-28b plasmid using the following standard procedures. (See, e.g., Sambrook, and Russell (2001), Molecular Cloning, A Laboratory Manual, 3th Ed.) The resulting construct is designated pksdD. (See, FIG. 1) The starting pET-28b plasmid is designed for direct cloning of heterologous genes in E. coli. The plasmid contains the highly expressed T7 promoter (base pair spacing between the trp-35 region and the lac UV5-10 region), the ribosome binding site, and an ATG initiation codon. Digestion with NdeI exposes the start codon for direct ligation and expression of foreign gene. The NdeI recognition sequence, commonly occurs at the start codon of prokaryotic genes, allowing direct ligation to the vector. (See, FIG. 2)

iii) Recombinant Hosts

E coli is considered to be one of the most useful recombinant hosts in expressing a heterologous protein. In accordance with the present invention, prednisolone produced by the recombinant host (e.g., E. coli) in the presence of hydrocortisone is devoid of contaminants (i.e., steroid-derived impurities). Without limited to a theory, the absence of steroid-derived impurities may be due to the fact that recombinant host such as E. coli lack the classes of enzymes including cytochrome P-450, mono-oxygenases, reductases, and the like which are responsible for the formation of steroid-derived impurities.

Accordingly, the present recombinant E. coli system constitutes an improved process leading to a production of prednisolone that is devoid of steroid-derived impurities. The present invention encompasses many commercially available strains of E. coli, many of which are derived from the same strain E coli K12. It would be generally considered that these commercially available strains are functionally equivalent for purposes of expressing a recombinant protein by one of skilled in the art. The commercially available strains include, but are not limited to, BL21 (DE3), BL21, DH5α, XL1-Blue, XL1-series, JM series and the like. More preferably, the bacterial host is BL-21 (DE3).

iv) Transformation of E. coli with ksdD Gene

For purposes of brevity and clarity, the following description is limited to a transformation construct containing a Δ¹-dehydrogenase gene. The identical procedure can be followed to insert other similar gene sequences that may have similar properties or activity, into a host to thereby enable or maximize the production of prednisolone.

Any E. coli strain can be transformed to contain the Δ¹-dehydrogenase (ksdD) gene insert described herein. The preferred strain is E. coli BL21 (DE3) (F⁻, ompT, hsdSB (r_{B}-m_{B}-) gal dcm (DE3), available commercially from Invitrogen (Carlsbad, Calif.). This strain was used as the host strain for prednisolone production in the Examples described below unless otherwise noted. Numerous suitable strains are commercially available from a host of commercial suppliers and the American Type Culture Collection.

For transformation of bacteria, a plasmid vector containing the gene insert is preferred. However, direct gene insertion into the E. coli genome can be used alternatively with standard methods. Several suitable vectors are available commercially or can be obtained by methods well known to the art. A preferred expression vector is pET-28b, available commercially from the (Novagen, Wis.). Suitable restriction enzymes and T4 DNA ligase to manipulate the vector can be obtained from several international suppliers, including Promega Corporation, (Madison, Wis.) and New England Biolabs (Beverly, Mass.).

Any E. coli strain can then be transformed using the pksdD construct. All transformations described herein were performed by the calcium chloride method using standard and well-known methodologies. While the calcium chloride method is preferred, transformation can be accomplished with equal success using any of several conventional procedures, such as electroporation and the like.

Microorganism Deposit

The E. coli BL21 (DE3) hosting the pksdD construct is designated as MSAF-1. The present bacterial strain MSAF-1, prepared and used for carrying out the examples, has been duly deposited on Apr. 4, 2006 at the Agricultural Research Service Culture Collection (NRRL) (B-30909), located at 1815 North University Street, Peoria, Ill. 61604 U.S.A., pursuant to the Budapest Treaty for the International Recognition of the Deposit of Microorganisms for the Purposed of Patent Procedure. All restrictions on the availability of the materials deposited will be irrevocably removed upon the issuance of a patent thereon.

iv) Fermentation

After successfully transformed with pksdD, the recombinant E. coli produces prednisolone using a fermentation process. Recombinant microorganisms having the Δ¹-dehydrogenase gene can be cultured in a conventional fermentation to produce prednisolone. The fermentation medium contains carbon sources, nitrogen sources, inorganic materials, amino acids, vitamins, etc, under aerobic conditions, while the temperature, pH, etc can be kept constant. The fermentation is carried out in the presence of hydrocortisone.

Carbon sources include, but not limited to, various carbohydrates such as glucose, fructose, sucrose, molasses, blackstrap molasses, hydrolyzates of starch, and the like; alcohols such as ethanol, glycerin, sorbitol, and the like; organic acids such as pyruvic acid, lactic acid, acetic acid, and the like; amino acids such as glycine, alanine, glutamic acid, aspartic acid, and the like. Any of the carbon sources may be used so long as the microorganisms can assimilate them. A concentration of the carbon sources is preferably 1 to 10%.

Nitrogen sources include, but not limited to, ammonia, various inorganic or organic ammonium salts such as ammonium chloride, ammonium sulfate, ammonium nitrate, ammonium carbonate, ammonium acetate, ammonium phosphate, and the like; amines; other nitrogen-containing compounds such as urea, NZ amine, meat extract, yeast extract, comsteep liquor, casein hydrolyzates, fish meal or its digested product, and the like. A concentration of the nitrogen sources is generally 0.1 to 10%.

Inorganic materials include, but not limited to, potassium dihydrogen phosphate, dipotassium hydrogen phosphate, magnesium phosphate, magnesium sulfate, sodium chloride, ferrous sulfate, zinc sulfate, calcium carbonate, and the like are used. If necessary and desired, amino acids, nucleic acids, vitamins, and the like required for growth of the microorganism may be supplemented to the medium.

The microorganism is cultured under aerobic conditions with shaking flasks or supplying oxygen by aeration and/or agitation in a mechanical fermentor. Temperature of the culture is preferably 20 to 40° C. The pH of the medium during the culture may be optionally maintained at about pH 7 with ammonia, urea, sodium hydroxide solution, and the like.

Preferably, the fermentation is performed for about 4 to about 140 hours. More preferably, the fermentation is performed for about 24 to about 96 hours. Most preferably, the fermentation is performed for about 60 hours.

In our specific embodiment, the fermentation broth includes the following ingredients: tryptone 10 grams/L, yeast extract 5 grams/L, and sodium chloride 10 grams/L. The broth is supplemented with the required antibiotic kanamycin in order to maintain the multiplication of the plasmid. One skilled in the art would know optimization of fermentation conditions to maximize production of prednisolone. For purposes of the present invention, increased prednisolone production in the host cell can be obtained using known methods.

v) Hydrocortisone

Hydrocortisone is conveniently used as a precursor for prednisolone production. However, the dehydrogenase reaction can also be performed on the hydrocortisone precursors. The hydrocortisone precursors include, but are not limited to cholesterol, pregnenolone, progesterone, 17α OH pregnenolone, 17α OH progesterone and 11-cortisol. Preferably, hydrocortisone is used at a concentration of about 0.1 to about 30 grams/L. More preferably, hydrocortisone is used at a concentration of about 1 to about 4 grams/L. Most preferably, hydrocortisone is used at a concentration of about 2 grams/L.

Hydrocortisone or its precursors may be added to the fermentation medium as an aqueous suspension or dissolved in suitable organic solvents. Preferred organic solvents include, but not limited to dimethylsulfoxide, methanol, ethanol, formamide, triethylene glycol and the like.

vi) Isolation of Prednisolone

Isolation of the prednisolone formed from the fermentation cell medium can be accomplished by any suitable means known in the separation art. Preferred isolation methods include filtering the fermentation culture medium to separate cells and cellular debris, followed by isolating the prednisolone from the fermentation medium by extraction followed by crystallization. (See, e.g., U.S. Pat. No.5,225,335). If so desired, the recombinant microorganisms may be completely lysed by suitable known means prior to the isolation of prednisolone.

Detailed Methodology

i) Extraction Protocol

Extraction of prednisolone from the fermentation broth is mediated by addition of ethyl acetate in a ratio of 1:1 (v/v). After shaking and phase separation in a separatory funnel the organic phase is collected. The extraction is repeated three times and the collected organic phases are combined and dried under vacuum. The dried extract is dissolved in methanol or acetonitrile and stored for HPLC analysis.

ii) HPLC Protocol

The samples are loaded in a HPLC instrument and separated in an Alltech C-1 8 analytical column, 5 μm, and 250 mm×4.6 mm. A mobile phase of 30% acetonitrile is used and the instrument is set up with an oven temperature of 35° C., a flow rate of 1 ml/min and the detection is at λ=244 nm.

iii) HPLC-MS Protocol

A Finnigan HPLC-MS equipped with an atmospheric pressure chemical ionization (APCI) chamber is used for molecular weight determination. The samples are separated using a LiChrospher 100 CN column (Merck), 5 μm, 250×4.6 mm. The flow rate is set up at 1 mL/min, the detection is carried out at 240 mn and a solution of 13% methanol is used as mobile phase. A positive polar ionization is used in the APCI chamber.

### EXAMPLES

Fermentation production of prednisolone was performed as described in Examples, below.

### Example 1

### Cloning of the Δ¹-dehydrogenase (EC 1.3.99.4) Gene

PCR Amplification

The Δ¹-dehydrogenase gene (ksdD gene) coding the 3-ketosteroid-1-dehydrogenase (Accession Number: D37969, Protein ID=BAA07186.1) from the microorganism Nocardioides simplex ATCC 6946 was used. A pair of PCR primers was designed to cover the open reading frame of the ksdD gene starting at the 1,435 bp position and ending at the 2,982 bp position. In addition, two restriction endonuclease sites corresponding to the restriction endonucleases NdeI and HindIII were added to the 5′ and 3′ ends, respectively. The sequences of the PCR primers are:

| | |
|---|---|
| sense | GGAATTCCATATGGACTGGGCAGAGGAG |

(underlined segment represents the recognition site of the endonuclease NdeI)

| | |
|---|---|
| anti-sense | AAAAAGCTTTCATCGCGCGTCCTCGG |

(underlined segment represents the recognition site of the endonuclease HindIII)

PCR reaction was prepared by mixing the following components in a sterile microtube (0.6 mL):

| | |
|---|---|
| Template DNA | 1 μl |
| sense oligonucleotide | 1 μl |
| anti-sense oligonucleotide | 1 μl |
| Eppendorf ® Master Mix | 10 μl |
| Water | 12 μl |

PCR reaction condition used was 1 cycle at 95° C. for 5 min, 30 cycles of 94, 55 and 72° C. for 45, 45 and 60 sec. respectively, 1 cycle at 72° C for 10 min and cooling at 4° C. until the reaction tube was either frozen or analyzed in an agarose gel. Genomic DNA from wild-type Nocardioides simplex (ATCC 6946) was extracted using the Wizard®Genomic kit (Promega, Madison, Wis., USA) and used as a DNA template.

Construction of Plasmid Hosting the KsdD Gene

Amplified ksdD gene was gel purified and ligated to the vector pET-28b to create plasmid pksdD. (See, FIGS. 1 and 2) Plasmid pET-28b was purchased from Novagen (Madison, Wis., USA) and linearized after digestion with NdeI/HindIII restriction enzymes. The DNA fragment corresponding to the ksdD gene was digested using the same restriction enzymes according to the following reaction:

| | |
|---|---|
| DNA fragment-ksdD gene | 16 μl |
| NdeI restriction enzyme | 1 μl (20 units/μl) |
| HindIII restriction enzyme | 1 μl (20 units/μl) |
| Buffer 2 | 2 μl |

Both restriction enzymes were purchased from New England Biolab, Beverly, Mass., USA. Buffer 2 is the recommended buffer for double digestion of DNA according to the supplier instructions. The ingredients were mixed in a sterile 1.5 ml-microtube and placed at 37° C. for 2 hours. After the digestion, both pET-28b and the ksdD gene were purified using Geneclean® II kit (Q-Biogene, Carlsbad, Calif.). The obtained purified DNA's were ligated according to the following reaction:

| | |
|---|---|
| pET-28b vector | 2 μl |
| ksdD gene | 7 μl |
| T4 Ligase | 1 μl |
| T4 Ligase buffer | 1.1 μl |

T4 ligase was purchased from New England Biolab, Beverly, Mass., USA. The ingredients were mixed in a sterile 1.5 mL microtube and placed at room temperature overnight.

### Example 2

### Transformation of pksdD Plasmid into E. coli BL21 (DE3)

The pksdD plasmid was transformed into E. coli strain BL21 (DE3) competent cells (Invitrogen (Carlsbad, Calif., USA)) via an overnight ligation reaction.

Competent Cell Preparation: The method used for competent cell preparation was adopted from Protocol 25 in: Molecular Cloning—A Laboratory Manual, Joseph Sambrook and David W. Russell, Third Edition, Vol. 1, Cold Spring Harbor Laboratory Press, NY, 2001, ppl. 116-1.118. Briefly, the strain was streaked on a petri plate containing solidified Luria-Bertani medium (LB) prepared as follows:

| | |
|---|---|
| Tryptone - Difco | 10 grams/L |
| Yeast extract - Difco | 5 grams/L |
| NaCl-EMD | 10 grams/L |

After dissolution of the solutes in 950 ml of deionized water, the volume was adjusted with the same liquid. Solidification of the medium was carried out by addition of 20 gram/liter of agar-EMD prior to sterilization. The mixture was sterilized by autoclaving for 20 min at 15 psi (1.05 kg/cm²) using a Tuttnauer-Brinkmann Model 3870-M autoclave.

A single colony was picked up from the streaked plate and placed in a sterile 10-ml glass tube containing 2 ml of sterile LB. The tube was shaken for 16-20 hours at 37° C. and 250 rpm. The culture was transferred into 100 ml of LB medium in a 1-liter flask. The culture was incubated for 3-4 hours at 37° C. and 250 rpm. The growth of the culture was monitored measuring its OD at a wavelength of λ=600 nm using a Bio-Mini Shimadzu spectrophotometer.

When the growing culture reached an OD_{λ=600} of 0.3-0.4, the culture was transferred into sterile, disposable, ice-cold 50-ml polypropylene tubes. After storing the tubes on ice for 10 min, the cells were recovered by centrifugation at 2700× g for 10 min at 4° C. The medium was discarded from the cell pellet and it was re-suspended in 30 ml of ice-cold MgCl₂—CaCl₂ solution (80 mM MgCl₂, 20 mM CaCl₂, 10% glycerol) by gentle vortexing. The cells were recovered again by centrifugation at 2700× g for 10 min at 4° C. The supernatant was discarded again and the cell pellet was re-suspended in 2 ml ice-cold MgCl₂—CaCl₂ solution for each 50 ml of original culture. The competent cell suspension was fractionated in aliquots of 100 μl and stored at −80° C. after rapid freezing using liquid nitrogen.

Chemical Transformation: A microtube containing 100 μl of frozen competent cells was thawed on ice. The complete ligation reaction was mixed with the thawed competent cells and placed on ice for 30 min. The tube was placed at 42° C. for 90 seconds and immediately returned to ice for 1 min. 900 μl of sterile LB broth was added to the mixture and incubated in a shaker for 60 min at 37° C. and 200 rpm. The content of the microtube was poured on a petri plate containing solidified LB medium supplemented with kanamycin at a final concentration of 50 μg/ml and incubated at 37° C. overnight.

Gene Insertion Confirmation: Three colonies were selected and picked separately up into a 10-ml sterile tube containing 2 ml of sterile LB broth supplemented with kanamycin at a final concentration of 50 μg/ml. The tubes were shaken for 16 hours at 37° C. and 200 rpm. The cultures were transferred to a 1.5 mL microtube and centrifuged in a Beckman-Coulter Microfuge® 18 Centrifuge at 6000× g for 10 min. The pksdD plasmid was extracted and purified using a PerfectPrep Plasmid Mini® (Eppendorf, Hamburg, Germany) kit. 20 μl of the extract were digested according to the following reaction:

| | |
|---|---|
| Plasmid DNA | 16 μl |
| NdeI restriction enzyme | 1 μl (20 units) |
| HindIII restriction enzyme | 1 μl (20 units) |
| Buffer 2 | 2 μl |

Both restriction enzymes were purchased from New England Biolab, Beverly, Mass., USA. Buffer 2 is the recommended buffer for double digestion of DNA when the restriction enzymes NdeI and HindIII are used according to the supplier instructions. The ingredients were mixed in a sterile 1.5-ml microtube and incubated at 37° C. for 2 hours. The vector pET-28b was digested in parallel using the same conditions. The resulting digestions were run on a 1% agarose gel at 120V.

### Example 3

### Fermentation Production of Prednisolone Using Wild-Type Nocardioides simplex (ATCC 6946): Biotransformation of Hydrocortisone (Comparative Studies)

A) Shake Flask Experiment: Wild type Nocardioides simplex (ATCC 6946) stored in cryovials in deep-freezer −80° C. was used for the fermentation process in shake flasks. The bioconversion fermentation process includes the following steps: i) growing a bacterial starter from a single colony; ii) diluting the bacterial starter (1:100); and iii) fermenting for prednisolone production.

Step i) Growth of a Bacterial Starter

Bacteria was streaked on a petri-dish containing the following media: 10 grams/L yeast extract, 15 grams/L glucose, 2 grams/L potassium hydrogen sulfate, 3 grams/L ammonium sulfate, and 100 mg/L magnesium chloride hexahydrate. The broth was solidified by the addition of 20 grams/L agar. The plate was placed in an incubator at 30° C. and stored at 4° C. when colonies were visible. A bacterial starter was initiated by picking a single colony up into a 125-ml flask containing 20 ml of sterile fresh medium and shaking for 15 hours at 30° C. and 250 rpm.

Step ii) Starter Dilution

0.2 ml of the bacterial starter was added (diluted 1:100) into a 125-ml flask containing 20 ml of fresh medium. The inoculated flask was shaken at 30° C. and 250 rpm for 8 hours.

Step iii) Fermentation for Prednisolone Production

Prednisolone production was initiated by addition of hydrocortisone (the substrate) as an aqueous suspension into the flask. The effects of substrate concentration and cell density on the steroid-derived impurity generation were evaluated. Hydrocortisone concentrations of 2, 5 and 20 grams/L were selected and added at different cell density (monitored as OD of the fermentation broth at λ=600 nm) in Table 1.

Monitoring of prednisolone and steroid-derived impurity formations were accomplished by sampling the flasks at different times followed by HPLC analysis.

Table 1 shows prednisolone and steroid-derived impurity production from a fermentation broth using wild-type Nocardioides simplex (ATCC 6946). These data demonstrate prednisolone production is accompanied by steroid-derived impurities during the conversion of hydrocortisone to prednisolone in fermentation using wild-type Nocardioides simplex. The amount of steroid-derived impurities present in the prednisolone preparation varies from about 5.1% to 20.6% of the total steroid (See, Table 1).

B) Fermentor Experiment

Wild-type Nocardioides simplex strain (ATCC 6946) stored in cryovials (2 ml) in deep freezer −80° C. was used for the fermentation process. Bacteria were directly seeded to a fermentor. The bioconversion fermentation process includes 3 steps: i) growth of bacteria; ii) induction of enzyme; and iii) prednisolone production.

Step i) Conditions for Growth of Biomass

A 8× diluted fermentation medium was prepared, the fermentation medium contained: 1.25 grams/L yeast extract, 1.875 grams/L glucose, 0.25 grams/L potassium hydrogen sulfate, 0.375 grams/L ammonium sulfate and 12.5 mg/L magnesium chloride hexahydrate. The diluted medium was found to be sufficient for bacteria growth and biotransforrnation. The use of diluted medium afforded adequate production of prednisolone and reduced level of by-products. Diluted fermentation medium was agitated by impellers (250 rpm), aerated (1.2 L sterile air/min) and maintained at a pH of 7.2 and a temperature of 32° C. for 14-16 hours.

Step ii) Induction Step

A hydrocortisone solution (200 mg of hydrocortisone in 20 ml methanol; final concentration 0.1 gram/L) was added into the diluted fermentation medium. The fermentation condition was maintained: agitation=500 rpm, aeration=1.2 L air/min, pH=7.2, temperature=32° C. and time=3 hours.

Step iii) Prednisolone Production

1.5 grams menadione sodium bisulfite (MBS) (an oxygen radical scavenger) as powder was added to achieve a final concentration of 2.7 mM. In addition to oxygen, MBS was the second component of reaction, which serves as artificial electron acceptor, instead the natural FADH/NADH, and contributes to proceeding of reaction. The MBS addition in combination of the 8× diluted medium provides an increase in initial reaction rate and prednisolone yield. Moreover, addition of MBS stabilizes maximum level of bioconversion.

When a concentration of >4 mM MBS was added, a high level (>4%) of prednisone was produced, accompanied by a low level (˜0-0.7%) and constant level of by-product (20β-hydroxyprednisolone). However, when a concentration of up to 2.7 mM MBS was added, prednisone was not detectable and there was a higher level of 20-hydroxy derivatives (>2.5%) being produced.

A hydrocortisone solution containing 4 grams of hydrocortisone, 4 grams CaCl₂ in 50 ml methanol (final concentration of 2 grams/L of hydrocortisone) was added. The fermentation condition was maintained: agitation=500 rpm, aeration=0.6 L air/min, pH=6.8, temperature=32° C. and time=5-7.5 hours.

Table 2 shows a fermentor experiment in which prednisolone and steroid-derived impurity production from a fermentation broth using wild-type Nocardioides simplex (ATCC 6946). These data confirm prednisolone production is accompanied by steroid-derived impurities during the larger-scale conversion of hydrocortisone to prednisolone in fermentation using wild-type Nocardioides simplex.

### Experiment 4

### HPLC-MS analysis of Fermentation Broth Using the Wild-Type Nocardioides simplex (ATCC 6946)

HPLC/MS was performed on a Finnigan System with an APCI chamber. HPLC analysis was carried out on a LiChrospher 100 CN, 4.6×250 mm-5 μm, column. An aqueous solution of 13% methanol at a flow rate of 1 ml/min was used. The column temperature was 23° C., and the UV detection wavelength was 254 nm. Atmospheric Pressure Chemical Ionization (APCI) was used setting up the instrument as following: capillary temperature 200° C., and an APCI vaporizer temperature 450° C. The samples were analyzed in positive scan mode with a mass range of 150-1200 Da.

HPLC separation and mass spectrometry analysis (MS) was performed to monitor the steroid-derived impurities of a fermentation broth using wild-type Nocardioides simplex (ATCC 6948) (See, FIGS. 3, 4 and 5). In this study, mass spectrometry analysis was focused to identify two of the steroid derived impurities: 20β-hydroxyhydrocortisone and 20β-hydroxyprednisolone (See, FIGS. 4 and 5).

A full scan acquisition was obtained showing the Total Ion Current Plot (TIC), in which two steroid-derived impurities with retention times at 13.40 (c) and 14.06 (d) are shown (arrows) (See, FIG. 4a)

A full scan acquisition was obtained showing the UV_{λ=254 nm} traces at the same retention times as in a) (See, FIG. 4b).

A full mass spectrometry was performed to monitor the steroid-derived impurity corresponding to the retention time range between 12.94 and 13.40. A molecular weight of 365.3 was observed, matching the molecular weight of 20β-hydroxy hydrocortisone. m/z indicates the detected ion peaks (m=molecular weight and z=number of protons). (See, FIG. 4c)

A full mass spectrometry was performed to monitor the steroid-derived impurity corresponding to the retention time range between 13.79 and 14.34. A molecular weight of 363.1 was observed, matching the molecular mass of 20β-hydroxy prednisolone. m/z indicates the detected ion peaks (m=molecular weight and z=number of protons) (See, FIG. 4d).

HPLC separation and mass spectrometry analysis (MS) was obtained showing the substrate (hydrocortisone), product (prednisolone) and one additional steroid-derived impurity of prednisone of a fermentation broth using wild-type Nocardioides simplex (ATCC 6948) (See, FIG. 4d)

HPLC separation and mass spectrometry analysis (MS) of the substrate (hydrocortisone), product (prednisolone) and one additional steroid-derived impurity of prednisone from a fermentation broth using wild-type Nocardioides simplex (ATCC 6948) (See, FIG. 5).

A full scan acquisition was obtained showing the Total Ion Current Plot (TIC), in which hydrocortisone (the substrate), prednisolone (the product) and a steroid-derived impurity with retention times at 16.30, 17.39 and 20.08 respectively are shown (arrows) (See, FIG. 5a).

A full scan acquisition was obtained showing the UV_{λ=254 nm} traces of the same compounds described in a) (See, FIG. 5b).

A full mass spectrometry was obtained showing the hydrocortisone corresponding to the retention time at 16.30. A molecular weight of 363.2 was observed, matching the molecular mass of hydrocortisone. m/z indicates the detected ion peaks (m=molecular weight and z=number of protons). (See, FIG. 5c)

A full mass spectrometry was obtained showing prednisolone that corresponds to the retention time at 17.38. A molecular weight of 361.1 was observed, matching the molecular mass of prednisolone. m/z indicates the detected ion peaks(m=molecular weight and z=number of protons) (See, FIG. 5d).

A full mass spectrometry was obtained showing the steroid-derived impurity corresponding to the retention time range between 19.55 and 20.52. A molecular weight of 359.1 was observed, matching the molecular mass of prednisone. m/z indicates the detected ion peaks(m=molecular weight and z=number of protons) (See, FIG. 5e).

Notwithstanding that wild-type bacteria are capable of converting hydrocortisone to prednisolone, the present data clearly show that the prepared prednisolone using wild-type Nocardioides simples is contaminated with steroid-derived impurities.

In another series of studies and using the same methodology as described in this Example, we have evaluated the conversion of hydrocortisone to prednisolone using wild-type Pseudomonas fluorescens (obtained from ATCC). Preliminary studies were performed using the shake flask system. After the fermentation, preliminary data revealed little prednisolone production and was accompanied by multiple steroid-derived impurities (data not shown).

### Example 5

### Fermentation Production of Prednisolone Using Recombinant E. coli: Biotransformation of Hydrocortisone

A) Shake Flask Experiment

Step i) Starter Preparation

A starting culture was prepared picking an E. coli strain BL-21 (DE3/pksdD) colony (i.e., MASF-1) into a 10 ml-glass tube containing 2 mL sterile LB medium and supplemented with 50 μg/ml kanamycin. The tube was shaken at 37° C. for 16 hours and 200 rpm.

Step ii) Inoculation and Induction

The starting culture was used for inoculation of 50 ml sterile LB medium supplemented with 50 μg/ml kanamycin contained in a 250 ml flask. Then, 500 μl of the starting culture was added into the fresh medium and shook at 37° C. until the OD_{λ=600} of the culture reached 1.2. At this moment, the temperature was reduced to 22° C. for 30 min and then isopropyl β-D-thiogalactoside (IPTG) was added at a final concentration of 100 μmol. Successively, hydrocortisone (substrate) at final concentrations of 2 and 10 mg/ml were added suspended in 2 ml ethanol. The flask was shaken for 72 hours at 22° C. and 200 rpm.

Step iii) Extraction and Analysis

The culture was extracted using 50 ml ethyl acetate (×3). The three fractions were combined and the solvent was volatilized in a Heidolph® evaporator model Laborota 4000. The extract was re-suspended in methanol and analyzed by HPLC. The HPLC analysis was carried out using a Shimadzu instrument and the steroids were separated in an analytical Econosil-C18 (Alltech) column, whose dimensions were 250 mm×4.6 mm. An aqueous solution of 30% acetonitrile was used as mobile phase and the steroids were detected at λ=244 nm. The column was kept in an oven at 35° C. and the flow rate was 1 ml/min.

Table 3 shows prednisolone and steroid-derived impurity production from fermentation broth using recombinant E. coli BL-21 (DE3) hosting the ksdD gene (Accession number D37969) (i.e., MASF-1).

These data clearly show that recombinant bacteria (e.g., MASF-1) that host a Δ¹-dehydrogenase gene are capable of converting hydrocortisone to prednisolone and that the obtained prednisolone is devoid of steroid-derived impurities. The amount of steroid-derived impurities was found to be below the detection limit (i.e., 0.01%).

B) Fermentor Experiment

Table 4 shows experiments carried out in a 2L-fermentor in which prednisolone and steroid-derived impurity production from fermentation broth using recombinant E. coli BL-21 (DE3) hosting the ksdD gene (Accession number D37969).

### Experiment 6

### HPLC-MS analysis from Fermentation Broth Using Recombinant E. coli: Biotransformation of Hydrocortisone

HPLC/MS was performed on a Finnigan System with an APCI chamber. HPLC analysis was carried out on a LiChrospher 100 CN, 4.6mm×250mm-5 μm. An aqueous solution of 13% methanol at a flow rate of 1 ml/min was used. The column temperature was 23° C., and the UV detection wavelength was 254 nm. Atmospheric Pressure Chemical Ionization (APCI) was used setting up the instrument as following: capillary temperature 200° C., APCI vaporizer temperature 450° C., and the samples were analyzed in positive scan mode with a mass range of 150-1200 Da.

HPLC separation and mass spectrometry analysis (MS) from fermentation broth using recombinant E. coli BL-21 (DE3)/pksdD (See, FIGS. 6 and 7).

A full scan acquisition showing the Total Ion Current Plot (TIC), in which two compounds with retention times at 12.13 (a) and 12.83 minutes (b) are shown (arrows) (See, FIG. 7a)

A full scan acquisition showing the UV_{λ=254 mm} traces at the same retention times as in a) (See, FIG. 7b).

A full mass spectrometry of the compound corresponding to the retention time at 12.13 minutes. A molecular weight of 361.1 was observed, matching the molecular weight of prednisolone. m/z indicates the detected ion peaks(m=molecular weight and z=number of protons) (See, FIG. 7c).

A full mass spectrometry of the corresponding to the retention time at 12.83 minutes. A molecular weight of 363.3 was observed, matching the molecular weight of hydrocortisone. m/z indicates the detected ion peaks(m=molecular weight and z=number of protons) (See, FIG. 7d).

These data clearly show that recombinant bacteria (e.g., MASF-1) hosting a Δ¹-dehydrogenase gene are capable of converting hydrocortisone to prednisolone that is devoid of steroid-derived impurities.

The disclosures of the cited publications are incorporated herein in their entireties by reference. While a few embodiments of the present invention have been shown and described, it is to be understood that many changes and modifications may be made thereunto without departing from the spirit and scope of the invention. The invention may be practiced other than as particularly described and still be within the scope of the accompanying claims.

TABLE 1
Shake Flask Experiments Prednisolone and steroid-derived impurity production from
fermentation broth using wild-type Nocardioides simplex (ATCC 6946)

| | | | | | | Steroid-derived Impurities | | |
|---|---|---|---|---|---|---|---|---|
| Hydrocortisone | | | | Hydrocortisone | Prednisolone | I | II | |
| added | | Time | | RT = 12.4 min | RT = 11.7 min | RT = 8.5 min | RT = 8.9 min | Total I + II |
| [g/L] | Experiment # | [hours] | OD_{λ=600 nm} | [%] | [%] | [%] | [%] | [%] |
| 2 | 1 | 1.5 | 1.3 | 95.2 | 0.1 | ND | 0.3 | |
| | | 4.5 | | 95.6 | 0.5 | ND | 0.3 | |
| | | 7.5 | | 95.6 | 1.0 | ND | 0.4 | |
| | | 32 | | 88.5 | 2.5 | ND | 1.3 | |
| | | 39 | | 92.9 | 3.5 | ND | 0.3 | |
| | | 57 | | 71.3 | 19.1 | 0.5 | 1.2 | |
| | | 81 | | 32.8 | 1.7 | 0.8 | 16.5 | 17.3 |
| | 2 | 1.5 | 3 | 96.6 | 0.1 | ND | 0.3 | |
| | | 4.5 | | 94.4 | 0.4 | ND | 0.3 | |
| | | 7.5 | | 96.2 | 0.6 | ND | 0.3 | |
| | | 32 | | 94.5 | 1.0 | ND | 0.7 | |
| | | 39 | | 93.0 | 3.4 | 0.01 | 0.3 | |
| | | 57 | | 79.0 | 16.5 | 0.1 | 0.4 | |
| | | 81 | | 36.7 | 46.5 | 4.2 | 3.8 | 8.0 |
| | 3 | 1 | 5 | 96.8 | 0.5 | ND | 0.3 | |
| | | 24 | | 91.7 | 2.8 | 0.2 | 0.8 | |
| | | 26 | | 89.8 | 4.5 | 0.2 | 0.8 | |
| | | 50 | | 24.5 | 65.8 | 4.2 | ND | |
| | | 74 | | 25.7 | 30.1 | 9.4 | 11.2 | 20.6 |
| 5 | 4 | 1.5 | 3 | 97.8 | 0.1 | ND | 0.4 | |
| | | 4.5 | | 97.6 | 0.2 | ND | 0.4 | |
| | | 7.5 | | 94.9 | 0.4 | ND | 0.6 | |
| | | 32 | | 94.4 | 2.1 | 0.01 | 0.3 | |
| | | 39 | | 96.3 | 1.7 | 0.01 | 0.3 | |
| | | 57 | | 89.1 | 8.8 | 0.04 | 0.4 | |
| | | 81 | | 53.5 | 38.0 | 2.6 | 1.4 | 4.0 |
| | 5 | 1 | 5 | 97.7 | 0.1 | ND | 0.3 | |
| | | 24 | | 94.9 | 1.4 | ND | 0.6 | |
| | | 26 | | 94.8 | 1.7 | ND | 0.6 | |
| | | 50 | | 72.2 | 23.3 | 0.7 | 0.6 | |
| | | 74 | | 16.1 | 72.0 | 3.9 | 2.3 | 6.2 |
| | 6 | 4.5 | 6.5 | 95.9 | 0.8 | ND | ND | |
| | | 7.5 | | 96.7 | 1.2 | ND | 0.3 | |
| | | 32 | | 13.9 | 74.2 | 4.7 | 2.5 | 7.2 |
| 20 | 7 | 16 | 3.6 | 78.1 | 11.6 | 0.8 | ND | |
| | | 24 | | 81.0 | 13.4 | 1.8 | 1.6 | |
| | | 44 | | 37.6 | 54.9 | 3.8 | 1.3 | 5.1 |

OD = Optical Density;
RT = Retention Time;
ND = Below detection sensitivity

TABLE 2
Fermentor Experiment Prednisolone and steroid-derived impurity production from
fermentation broth using wild-type Nocardicides simplex (ATCC 6946)

| | | | | | Steroid-derived Impurities | |
|---|---|---|---|---|---|---|
| Hydrocortisone | | | Hydrocortisone | Prednisolone | 20β-OH prednisolone | Prednisone |
| added | | Time | RT = 16.30 min | RT = 17.40 min | RT = 14.05 min | RT = 20.10 min |
| [g/L] | Experiment # | [hours] | [%] | [%] | [%] | [%] |
| 2 | 1 | 7 | 1.8 | 94.8 | 3.4 | ND |

RT = Retention Time;
ND = Below detection sensitivity

TABLE 3
Shake Flask Experiments Prednisolone and steroid-derived impurity production from fermentation broth using
recombinant E. coli BL-21 (DE3) hosting a ksdD gene (SEQ ID NO. 1; Accession Number:
D37969)

| | | | | | | | | | | Steroid-derived | |
|---|---|---|---|---|---|---|---|---|---|---|---|
| | | | | | | | | | | Impurities | |
| Hydro- | | | | | | | | | | I | II |
| cortisone | | | | Conversion | IPTG | Induction | | Hydrocortisone | Prednisolone | RT = 8.5 | RT = 8.9 |
| added | Experiment | Time | | Temperature | added | Time | Steroid | RT = 12.4 min | RT = 11.7 | min | min |
| [g/L] | # | [hours] | OD_{λ=600 nm} | [° C.] | [μmol] | [hours] | solvent | [%] | min [%] | [%] | [%] |
| 2 | 1 | 96 | 2.5 | 25 | 200 | 0 | water | 35.9 | 57.9 | ND | ND |
| | 2 | 20 | 2.0 | 22 | 500 | 15 | water | 84.1 | 11.2 | ND | ND |
| | | 120 | | | | | | 63.7 | 31 | ND | ND |
| | 3 | 96 | 2.3 | 25 | 1000 | 0 | water | 69.3 | 26.9 | ND | ND |
| | 4 | 24 | 2.3 | 22 | 200 | 72 | DMSO | 82.5 | 7.4 | ND | ND |
| | | 48 | | | | | | 79.3 | 9.8 | ND | ND |
| | | 72 | | | | | | 75.3 | 12.7 | ND | ND |
| 10 | 5 | 14 | 2.5 | 22 | 200 | 0 | water | 97.0 | 0.4 | ND | ND |
| | | 64 | | | | | | 94.7 | 1.5 | ND | ND |
| | | 136 | | | | | | 95.0 | 2.5 | ND | ND |
| | 6 | 14 | 2.3 | 22 | 500 | 0.5 | DMSO | 90.8 | 4.6 | ND | ND |
| | | 62 | | | | | | 85.5 | 5.7 | ND | ND |
| | | 86 | | | | | | 83.7 | 7.5 | ND | ND |
| | 7 | 14 | 2.3 | 22 | 200 | 16 | DMSO | 95.9 | 1.0 | ND | ND |
| | | 38 | | | | | | 89.5 | 4.8 | ND | ND |
| | | 62 | | | | | | 88.9 | 6.4 | ND | ND |
| | | 134 | | | | | | 86.2 | 9.4 | ND | ND |

OD = Optical Density;
RT = Retention Time;
ND = Below detection sensitivity;
DMSO = dimethylsulfoxide.

TABLE 4
Fermentor Experiments Prednisolone and steroid-derived impurity production from fermentation using
recombinant E. coli BL-21 (DE3) hosting a ksdD gene (SEQ ID NO. 1; Accession Number: D37969)

| | | | | | | | | | | Steroid-derived | |
|---|---|---|---|---|---|---|---|---|---|---|---|
| | | | | | | | | | | Impurities | |
| Hydro- | | | | | | | | | | I | II |
| cortisone | | | | Conversion | IPTG | Induction | | Hydrocortisone | Prednisolone | RT = 8.5 | RT = 8.9 |
| added | Experiment | Time | | Temperature | added | Time | Steroid | RT = 12.4 min | RT = 11.7 | min | min |
| [g/L] | # | [hours] | OD_{λ=600 nm} | [° C.] | [μmol] | [hours] | solvent | [%] | min [%] | [%] | [%] |
| 2 | 1 | 0 | 4.6 | 22 | 200 | 0 | water | 95.9 | 0.3 | ND | ND |
| | | 3 | | | | | | 95.4 | 0.7 | ND | ND |
| | | 18 | | | | | | 89.4 | 6.6 | ND | ND |
| | | 22 | | | | | | 88.9 | 7.0 | ND | ND |
| | | 26 | | | | | | 87.6 | 9.0 | ND | ND |
| | | 43 | | | | | | 86.5 | 10.3 | ND | ND |
| | | 50 | | | | | | 84.1 | 12.5 | ND | ND |
| | | 67 | | | | | | 80.9 | 16.0 | ND | ND |
| | 2 | 2 | 6.4 | 25 | 1000 | 0 | water | 96.3 | 1.4 | ND | ND |
| | | 4 | | | | | | 93.1 | 1.7 | ND | ND |
| | | 6 | | | | | | 93.1 | 2.4 | ND | ND |
| | | 8 | | | | | | 90.9 | 3.5 | ND | ND |
| | | 25 | | | | | | 85.9 | 8.7 | ND | ND |
| | | 27 | | | | | | 81.0 | 13.1 | ND | ND |
| | | 31 | | | | | | 82.1 | 13.8 | ND | ND |
| | | 73 | | | | | | 74.6 | 21.5 | ND | ND |
| | 3 | 1 | 6.4 | 30 | 1000 | 0 | water | 89.9 | 1.6 | ND | ND |
| | | 4 | | | | | | 89.2 | 4.8 | ND | ND |
| | | 6 | | | | | | 88.2 | 7.9 | ND | ND |
| | | 8 | | | | | | 85.7 | 9.2 | ND | ND |
| | | 25 | | | | | | 79.9 | 15.3 | ND | ND |
| | | 33 | | | | | | 64.9 | 28.6 | ND | ND |
| | 4 | 1 | 3.9 | 21 | 200 | 2 | water | 95.9 | 0.3 | ND | ND |
| | | 19 | | | | | | 90.8 | 4.7 | ND | ND |
| | | 44 | | | | | | 89.3 | 7.6 | ND | ND |
| | | 72 | | | | | | 70.0 | 24.8 | ND | ND |
| | 5 | 39 | 3.9 | 21 | 1000 | 0 | water | 90.3 | 3.5 | ND | ND |
| | | 48 | | | | | | 86.6 | 3.3 | ND | ND |
| | | 63 | | | | | | 90.2 | 3.3 | ND | ND |
| | | 71 | | | | | | 90.4 | 3.0 | ND | ND |
| | | 84 | | | | | | 90.4 | 4.7 | ND | ND |
| | 6 | 4 | 6.25 | 22 | 500 | 0 | water | 94 | 1.7 | ND | ND |
| | | 22 | | | | | | 92.6 | 3.2 | ND | ND |
| | | 27 | | | | | | 92.3 | 3.6 | ND | ND |
| | | 42 | | | | | | 92.6 | 3.4 | ND | ND |
| | 7 | 1 | 3.7 | 22 | 200 | 0 | water | 96.6 | 0.1 | ND | ND |
| | | 18 | | | | | | 92.7 | 3.8 | ND | ND |
| | | 25 | | | | | | 90.7 | 5.0 | ND | ND |
| | | 41 | | | | | | 88.7 | 7.7 | ND | ND |
| | 8 | 1 | 3.6 | 22 | 200 | 2 | water | 94.5 | 1.4 | ND | ND |
| | | 9 | | | | | | 91.0 | 4.8 | ND | ND |
| | | 24 | | | | | | 86.4 | 9.3 | ND | ND |
| | | 68 | | | | | | 80.4 | 15.2 | ND | ND |
| | 9 | 6 | 3.5 | 24 | 200 | 0 | DMSO | 93.2 | 0.2 | ND | ND |
| | | 52 | | | | | | 67.2 | 20.6 | ND | ND |
| | | 78 | | | | | | 60.7 | 29.3 | ND | ND |

OD = Optical Density;
RT = Retention Time;
ND = Below detection sensitivity;
DMSO = dimethylsulfoxide.

What is claimed is:

1. A process for preparing prednisolone, comprising the steps of:
(a) preparing an E. coli transformed with a recombinant DNA molecule which is a plasmid containing a nucleic acid sequence set forth in SEQ ID No: 1;
(b) culturing the transformed E. coli in a fermentation medium containing hydrocortisone; and
(c) recovering prednisolone from the fermentation medium,
wherein the recovered prednisolone is devoid of steroid-derived impurity.

2. The process of claim 1, wherein the transformed E. coli is transformed with a recombinant DNA molecule that encodes a polypeptide comprising the amino acid sequence set forth in SEQ ID No: 2.

3. The process of claim 1, wherein the plasmid is pksdD.

4. The process of claim 1, wherein the nucleic acid sequence is a dehydrogenase gene of a bacterial source selected from Nocardioides simplex (Accession number: D37969), Pseudomonas testeroni, Pseudomonas fluorescens, and Mycobacterium smegmatis.

5. The process of claim 1, wherein the nucleic acid sequence is a dehydrogenase gene of Nocardioides simplex (Accession number: D37969).

6. The process of claim 1, wherein the E. coli is a microorganism selected from the group consisting of BL-21 (DE3), BL-21, DH5α, XL1-Blue, XL1-series, and JM series.

7. The process of claim 1, wherein the E. coli is BL-21 (DE3).

8. The process of claim 1, wherein the transformed E. coli is MSAF-1.

9. The process of claim 1, wherein the culturing step is performed in a fermentation.

10. The process of claim 9, wherein the fermentation is performed for about 4 to about 140 hours.

11. The process of claim 9, wherein the fermentation is performed for about 24 hours to about 96 hours.

12. The process of claim 9, wherein the fermentation is performed for about 60 hours.

13. The process of claim 1, wherein the fermentation medium contains hydrocortisone at a concentration of about 0.1 to about 30 grams/L.

14. The process of claim 1, wherein the fermentation medium contains hydrocortisone at a concentration of about 1 to about 4 grams/L.

15. The process of claim 1, wherein the fermentation medium contains hydrocortisone at a concentration of about 2 grams/L.

16. The process of claim 1, wherein the recovered prednisolone contains less than about 1% total steroid-derived impurity.

17. The process of claim 1, wherein the recovered prednisolone contains less than about 0.5% total steroid-derived impurity.

18. The process of claim 1, wherein the recovered prednisolone contains less than about 0.1% total steroid-derived impurity.

19. The process of claim 1, wherein the recovered prednisolone contains less than about 0.01% total steroid-derived impurity.

20. The process of claim 1, wherein the steroid-derived impurity is at least one compound selected from the group consisting of 20β-hydroxy derivative of prednisolone, 11α-hydroxy derivative of prednisolone, 11-oxo derivative prednisolone and 11-deoxy of prednisolone.

21. The process of claim 20, wherein the steroid-derived impurity is 20β-hydroxy derivative of prednisolone.

22. The process of claim 20, wherein the steroid-derived impurity is 11α-hydroxy derivative of prednisolone.

23. The process of claim 20, wherein the steroid-derived impurity is 11-oxo derivative prednisolone.

24. The process of claim 20, wherein the steroid-derived impurity is 11-deoxy of prednisolone.

25. The process of claim 20, wherein the E. coli converts hydrocortisone to prednisolone.

26. Prednisolone having less than about 1% total steroid-derived impurity prepared by a process which comprises the steps of:
(a) preparing an E. coli transformed with a recombinant DNA molecule which is a plasmid containing a nucleic acid sequence set forth in SEQ ID No: 1;
(b) culturing the transformed E. coli in a fermentation medium containing hydrocortisone; and
(c) recovering prednisolone from the fermentation medium.
